(19) United States
(12) Patent Application Publication
(10) Pub. No.: US 2018/0250320 A1
(43) Pub. Date: Sep. 6, 2018

(54) COMPOSITIONS AND METHODS FOR TREATING SOLID TUMORS

(71) Applicant: THE JOHNS HOPKINS UNIVERSITY, Baltimore, MD (US)

(72) Inventors: Cynthia Zahnow, Bel Air, MD (US); Stephen B. Baylin, Baltimore, MD (US); Michael Topper, Baltimore, MD (US); Meredith Stone, Baltimore, MD (US)

(21) Appl. No.: 15/755,310

(22) PCT Filed: Aug. 26, 2016
(86) PCT No.: PCT/US16/48933

§ 371 Date: Feb. 26, 2018

Related U.S. Application Data
(60) Provisional application No. 62/209,956, filed on Aug. 26, 2015.

(51) Int. Cl. A61K 31/706; C07K 16/28; A61P 35/00; A61K 45/06

(52) U.S. Cl. CPC A61K 31/706; C07K 16/2818; C07K 16/2827; A61K 31/4406; A61K 45/06; A61K 2300/00; A61P 35/00

(57) ABSTRACT

The present invention relates to the field of cancer. More specifically, the present invention provides compositions and methods useful for treating solid tumors. In a specific embodiment, a method for treating a solid tumor in a patient having cancer comprises the step of administering to the patient a demethylating agent, a histone deacetylase (HDAC) inhibitor and a checkpoint inhibitor.

### CROSS-REFERENCE TO RELATED APPLICATIONS

This application claims the benefit of U.S. Provisional Application No. 62/209,956, filed Aug. 26, 2015, which is incorporated herein by reference in its entirety.

### STATEMENT OF GOVERNMENTAL INTEREST

This invention was made with U.S. government support under grant no. W81XWH-13-OCRP-TIA. The U.S. government has certain rights in this invention.

### FIELD OF THE INVENTION

The present invention relates to the field of cancer. More specifically, the present invention provides compositions and methods useful for treating solid tumors.

### BACKGROUND OF THE INVENTION

Cancers are now recognized as being driven by widespread changes in the epigenome including changes in DNA methylation and chromatin packaging. Changes in DNA methylation include global loss of methylation and focal gain of methylation at promoter regions of tumor suppressor genes leading to transcriptional silencing. DNA methylation, the covalent modification of DNA, is mediated by a family of DNA methyltransferases (DNMTs). In recent years, inhibitors of DNMTs (DNMTis) have emerged as therapeutic targets for treatment of myeloid malignancies as well as cutaneous T cell lymphoma. In 2004, the FDA approved the DNMT inhibitor 5-azacitidine (AZA) for treatment of myelodysplastic syndrome. However, new and better therapies for treating cancer are desperately needed.

### SUMMARY OF THE INVENTION

The present invention is based, at least in part, on the discovery that solid tumors can be treated with a combination therapy of a demethylating agent, a histone deacetylase (HDAC) inhibitor and a checkpoint inhibitor.

Thus, in one aspect, the present invention provides methods for treating solid tumors in patients. Any solid tumor is contemplated including, but not limited to, ovarian, breast, melanoma, lung, colon, pancreas, liver, esophageal, stomach, epithelial, sarcoma, cervical, and uterine. In certain embodiments, the cancer is ovarian, breast or melanoma. The methods comprise a combination therapy comprising administration of a demethylating agent, an HDAC inhibitor and/or a checkpoint inhibitor. In particular embodiments, the method comprises administration of a demethylating agent and an HDAC inhibitor. In other embodiments, the method comprises administering a demethylating agent, an HDAC inhibitor and a checkpoint inhibitor. In certain embodiments, the methods may further comprise priming the patient with a prior administration of a demethylating agent, and then administering the combination therapy. Thus, in one embodiment, a methods for treating a solid tumor in a patient comprises the steps of (a) administering a demethylating agent; and (b) administering a demethylating agent, HDAC inhibitor and a checkpoint inhibitor.

In particular embodiments, the demethylating agent comprises, but is not limited to, 5-azacytidine (AZA), 5-azadeoxycytidine (DAC) SGI-110 (guadecitabine) or analogs of the foregoing. In a specific embodiment, the demethylating agent comprises AZA. In other embodiments, the HDAC inhibitor comprises, but is not limited to, givinostat, entinostat or analogs thereof. In further embodiments, the checkpoint inhibitor comprises, but is not limited to, an anti-PD1 antibody (e.g., nivolumab, pembrolizumab (keytruda)), an anti-PDL-1 antibody (e.g., Medi4736) or an anti-CTLA4 antibody (e.g., tremelimumab).

In yet another embodiment, a method for treating a solid tumor in a patient comprises the step of administering (a) AZA or DAC; (b) givinostat or entinostat; and (c) nivolumab, pembrolizumab (keytruda), Medi4736, MPDL3280A or tremelimumab.

In another aspect, the present invention provides compositions for treating solid tumors in patients. In one embodiment, a composition comprises a demethylating agent and an HDAC inhibitor. In a further embodiment, the composition further comprises a checkpoint inhibitor. In particular embodiments, the demethylating agent is AZA, DAC or guadecitabine. In other embodiments, the HDAC inhibitor is givinostat or entinostat. In a specific embodiment, the demethylating agent is AZA or DAC and the HDAC inhibitor is givinostat or entinostat. In another embodiment, a pharmaceutical composition comprises a demethylating agent, an HDAC inhibitor and a checkpoint inhibitor. In a specific embodiment, the pharmaceutical composition comprises AZA and givinostat. In further embodiments, the checkpoint inhibitor is an anti-PD1 antibody, an anti-PDL-1 antibody or an anti-CTLA4 antibody.

### BRIEF DESCRIPTION OF THE FIGURES

FIGS. 1A and 1B. Treatment with AZA, anti-PD-1, and HDAC inhibitors improves survival in the ID8 model of ovarian cancer.

FIGS. 2A and 2B. AZA treatment combined with HDAC inhibitors improves survival in the ID8 model of ovarian cancer.

FIG. 3. AZA treatment combined with anti-PD-1 and HDAC inhibitors, or HDAC inhibitors combined with anti-PD-1, decreases the amount of ascites fluid developed.

FIGS. 4A and 4B. HDAC inhibitors (Givinostat-ITF-green line) and (Entinostat-MS275-blu line) increase expression of anti-viral genes (FIG. 4A) and cancer testis antigens (FIG. 4B) when used in combination with AZA, but NOT when used as single agents. Human lung cancer cell lines H23 and A549 were primed by treatment with AZA (red line) for 5 days followed by 3-5 days of treatment with the HDACi. For many genes, addition of an HDACi to AZA increases gene expression above levels attained by AZA alone.

FIG. 5. In vivo treatment of mice with epigenetic therapy leads to an overall increase in the number of CD3 T cells recruited to the ovarian cancer associated ascites. CD3 T cells were calculated as a percentage of total CD45 cells (which marks all immune cells).

FIG. 6. Activated CD4 Cells (CD4+IFNG+) are increased in the ascites of mice with ovarian cancer that are treated with Aza+HDACis+anti-PD-1. CD4+IFNG+ can be T-helpers (Th1). Ascites cells were plated and treated with PMA and ionomycin for 4 hrs to stimulate the CD3 T cell receptor. CD45 cells were analyzed by flow.

FIG. 7. Interferon gamma positive CD8 cells are increased (CD8+IFNG+) in the ascites of mice with ovarian cancer that are treated with Aza+HDACis+anti-PD-1. These cells represent CD8 Teff cells that would attack tumor cells. Ascites cells were plated and treated with PMA and ionomycin for 4 hrs to stimulate the CD3 T cell receptor. CD45 cells were analyzed by flow.

FIG. 8. Epigenetic therapy decreases the MDSCs in the ascites of mice with ovarian cancer. These are the GR-1+CD11b+ cells in the CD45 immune cell population that are also negative for MHCII and F4/80. MDSCs facilitate an immuno-suppressive microenvironment for the tumor. Ascites cells were plated and treated with PMA and ionomycin for 4 hrs to stimulate the T cells. CD45 cells were analyzed by flow.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

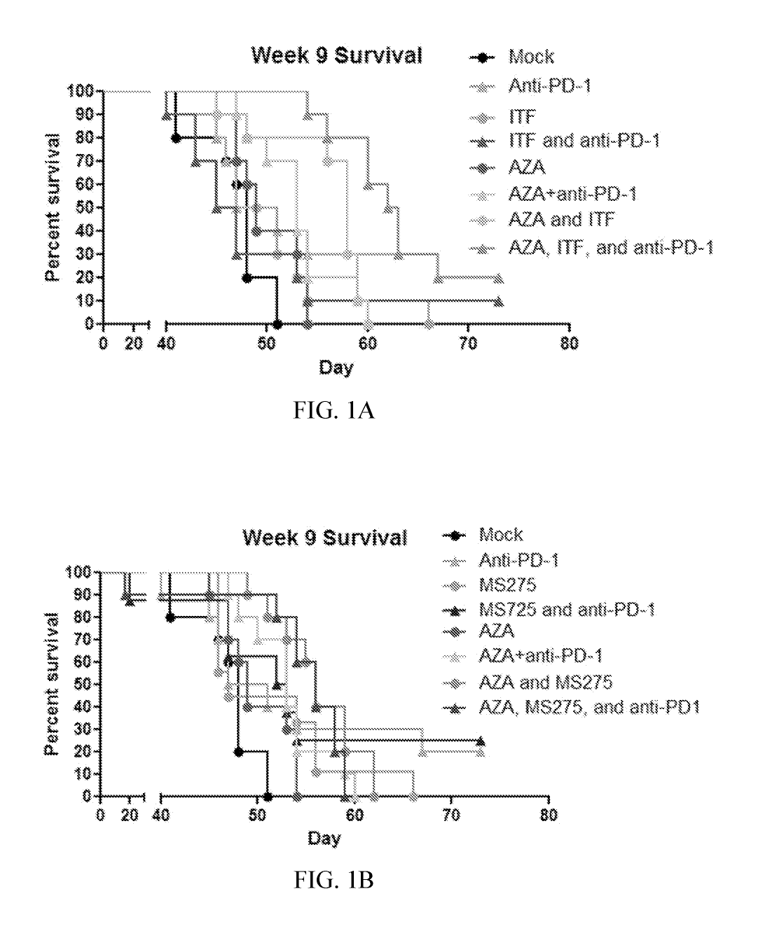

The present inventors have discovered that solid tumors can be treated with a combination therapy of a demethylating agent, an HDAC inhibitor and a checkpoint inhibitor. As shown in FIG. 1, mice treated with 5-Azacytidine (AZA) combined with anti-PD-1 and with HDAC inhibitors entinostat (MS275) or givinostat (ITF) survived longer than untreated mice or mice given a single agent. Mice were injected with 2.5×10⁶ VEGF-defensin ID8 ovarian cells i.p. Three days later, mice began treatment with 0.5 mg/kg 5-azacytidine (AZA) for 5 days per week. The next week the mice were treated with either 2 mg/kg MS275 or ITF 5 days a week, and the treatments alternated by week until the end of the experiment. Doses of 200 μg anti-PD-1 were given on day 17, 20, 24, and 27 after injection. FIG. 1A shows survival of mice treated with AZA, anti-PD-1 and ITF. AZA combined with one or two other agents significantly improves survival. For the triple combination, p<0.0001. FIG. 1B shows survival of mice treated with AZA, anti-PD-1 and MS275. MS275 and anti-PD-1 significantly improved survival compared to Mock (p=0.0315). AZA combined with any other agent significantly improved survival, and with three treatments compared to Mock (p<0.0002).

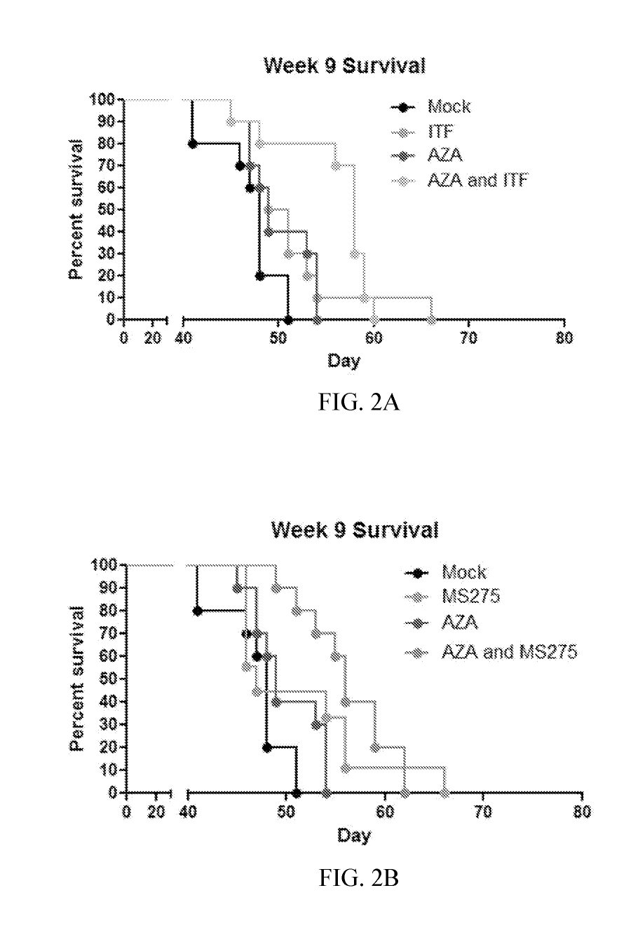

FIG. 2 shows the results of experiments in which mice treated with AZA combined with HDAC inhibitors entinostat (MS275) or givinostat (ITF) survived longer than untreated mice or mice given a single agent. Mice were injected with 2.5×10⁶ VEGF-defensin ID8 ovarian cells i.p. Three days later, mice began treatment with 0.5 mg/kg 5-azacytidine (AZA) for 5 days per week. The next week the mice were treated with either 2 mg/kg MS275 or ITF 5 days a week, and the treatments alternated by week until the end of the experiment. FIG. 2A shows survival of mice treated with AZA and ITF. The combination treatment significantly improves survival (p=0.0006). FIG. 2B shows survival of mice treated with AZA and MS275. The combination treatment significantly improved survival (p<0.0001).

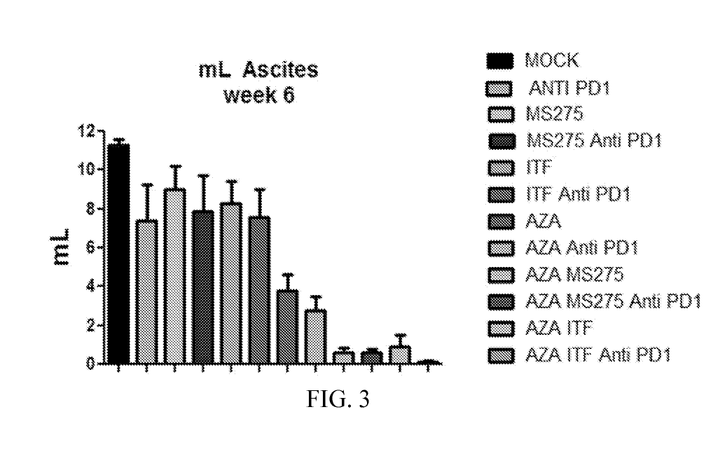

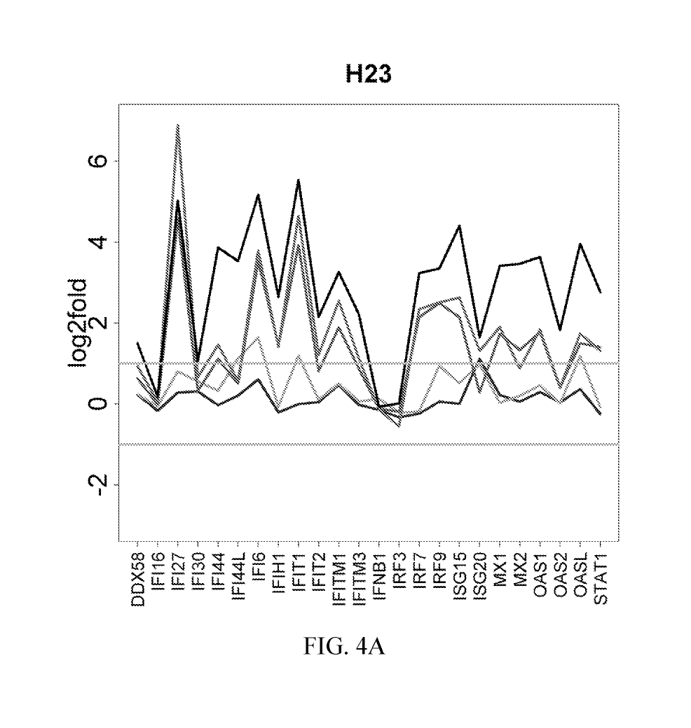

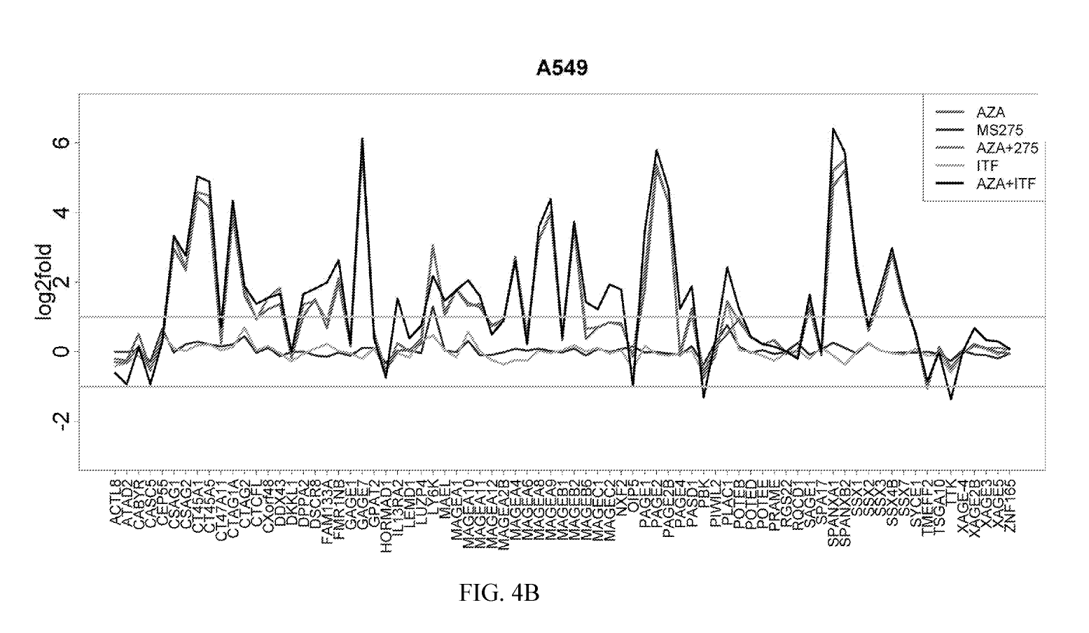

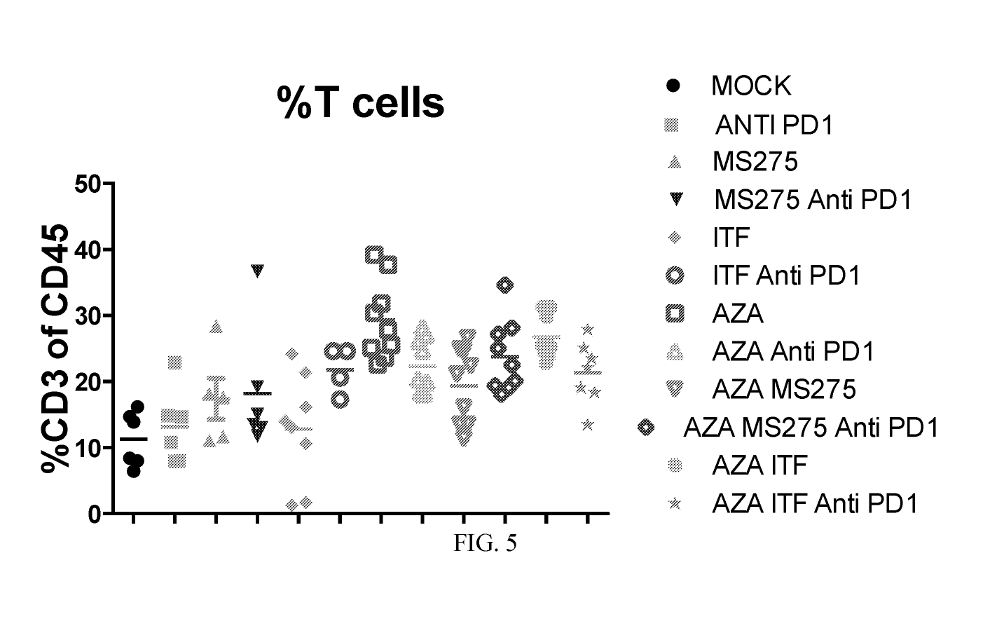

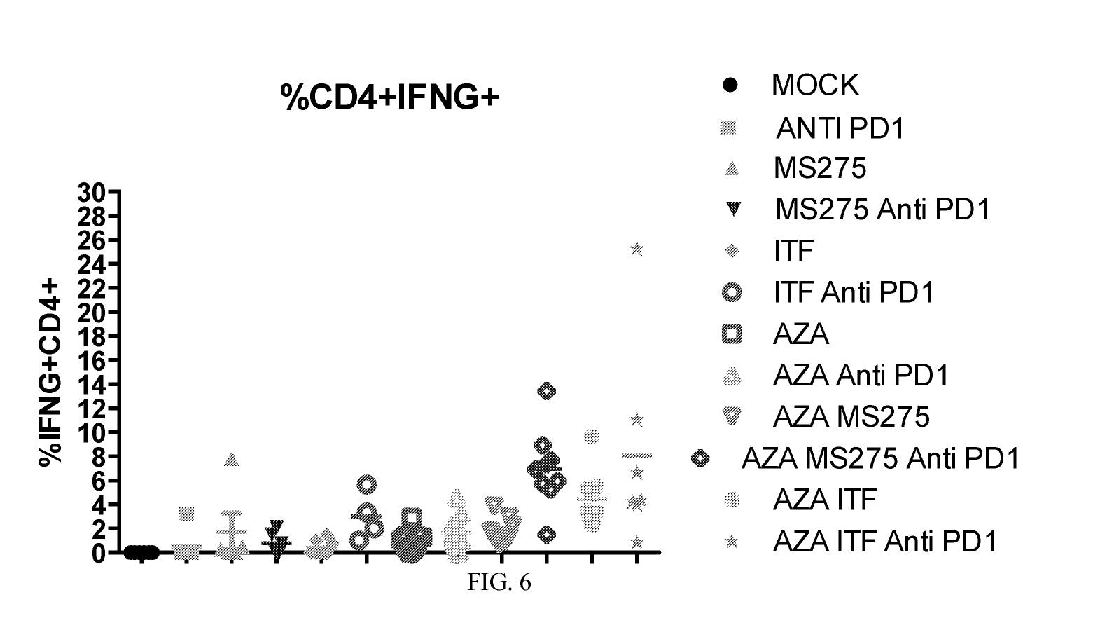

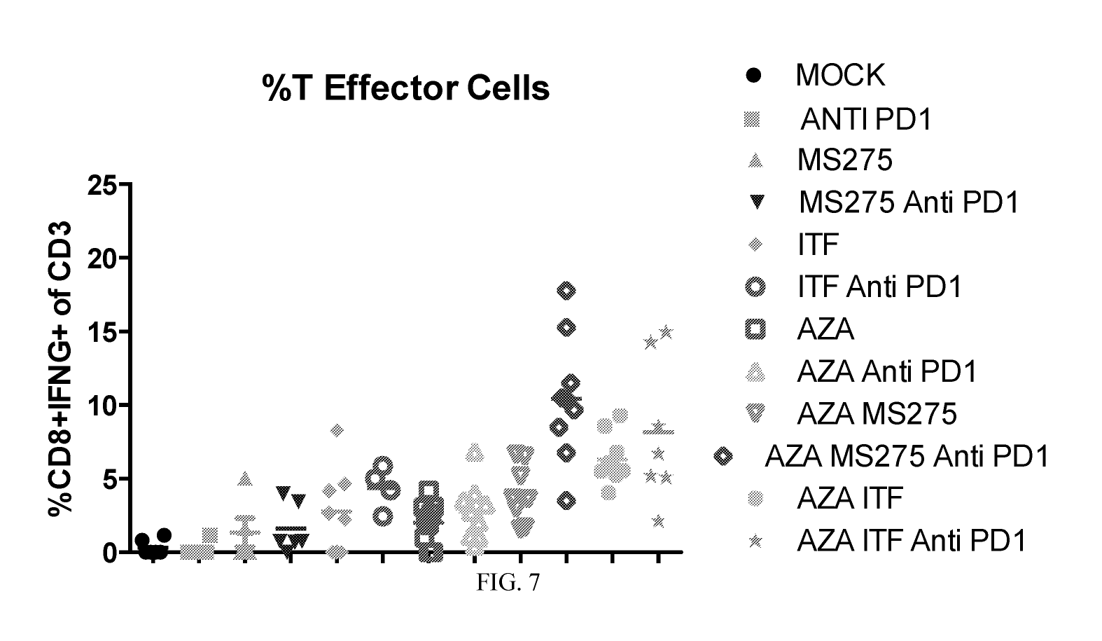

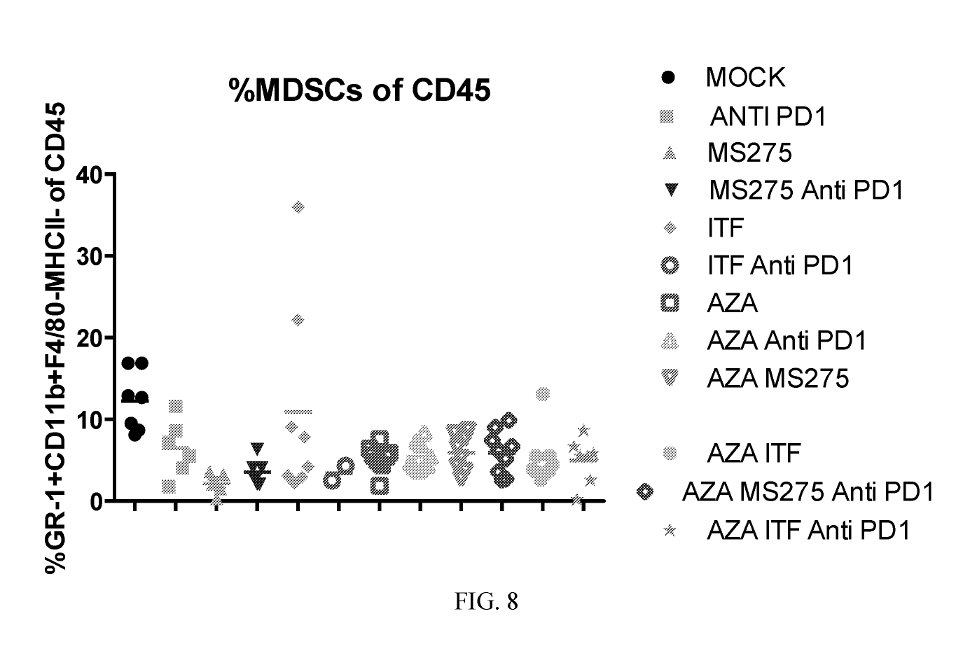

FIG. 3 shows that mice treated with 5-Azacytidine (AZA) combined with anti-PD-1 and HDAC inhibitors entinostat (MS275) or givinostat (ITF) developed less ascites fluid than untreated or mice given a single agent. Mice were injected with 2.5×10⁶VEGF-defensin ID8 ovarian cells i.p. Three days later, mice began treatment with 0.5 mg/kg 5-azacytidine (AZA) for 5 days per week. The next week the mice were treated with either 2 mg/kg MS275 or ITF 5 days a week, and the treatments alternated by week until the end of the experiment. Mice developed ascites fluid by week 4.5, and were drained and the ascites volume was measured. Ascites volume from mice drained at week 6 after injection. Mice treated with anti-PD-1 and ITF or Givinostat had significantly less ascites than untreated mice. Furthermore, mice treated with AZA alone or with any combination with AZA also had significantly reduced ascites volume compared to Mock.

### I. Definitions

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present. The term “inhibitor” is synonymous with the term antagonist.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., a demethylating agent, HDAC inhibitor and/or immunotherapy. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of treating solid tumors in patients.

As used herein, the term “effective,” means adequate to accomplish a desired, expected, or intended result. More particularly, an “effective amount” or a “therapeutically effective amount” is used interchangeably and refers to an amount of demethylating agent, HDAC inhibitor and/or checkpoint inhibitor necessary to provide the desired “treatment” (defined herein) or therapeutic effect, e.g., an amount that is effective to prevent, alleviate, treat or ameliorate symptoms of a disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The term “combination” refers to two or more therapeutic agents to treat a condition or disorder described herein. Such combination of therapeutic agents may be in the form of a single pill, capsule, or intravenous solution. However, the term “combination” also encompasses the situation when the two or more therapeutic agents are in separate pills, capsules, syringes or intravenous solutions. Likewise, the term “combination therapy” refers to the administration of two or more therapeutic agents to treat a therapeutic condition or disorder described herein. Such administration encompasses co-administration of these therapeutic agents in a substantially simultaneous manner, such as in a single capsule having a fixed ratio of active ingredients or in multiple, or in separate containers (e.g., pills, capsules, etc.) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The terms “co-administration” and “in combination with” include the administration of two or more therapeutic agents simultaneously, concurrently or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

The term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in vivo) to provide the compound. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human or animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

As used herein, and unless otherwise specified, the terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### II. Demethylating Agents

DNA demethylating agents useful in the methods provided herein include, but are not limited to, 5-azacytidine (azacytidine), 5-azadeoxycytidine (decitabine; DAC), SGI-110 (guadecitabine) zebularine and procaine. In one embodiment, the DNA demethylating agent is 5-azacytidine. 5-azacitidine is 4-amino-1-β-D-ribofuranozyl-s-triazin-2(1H)-one, also known as VIDAZA®. Its empirical formula is C₈H₁2N₄O₅, the molecular weight is 244. 5-azacitidine is a white to off-white solid that is insoluble in acetone, ethanol and methyl ketone; slightly soluble in ethanol/water (50/50), propylene glycol and polyethylene glycol; sparingly soluble in water, water-saturated octanol, 5% dextrose in water, N-methyl-2-pyrrolidone, normal saline and 5% Tween 80 in water, and soluble in dimethylsulfoxide (DMSO).

In one embodiment, the methods provided herein comprise administration or co-administration of one or more DNA demethylating agents. In one embodiment, the DNA demethylating agents are cytidine analogs. A cytidine analog referred to herein is intended to encompass the free base of the cytidine analog, or a salt, solvate, hydrate, cocrystal, complex, prodrug, precursor, metabolite, and/or derivative thereof. In certain embodiments, a cytidine analog referred to herein encompasses the free base of the cytidine analog, or a salt, solvate, hydrate, cocrystal or complex thereof. In certain embodiments, a cytidine analog referred to herein encompasses the free base of the cytidine analog, or a pharmaceutically acceptable salt, solvate, or hydrate thereof.

In certain embodiments, the cytidine analog is 5-azacytidine (5-azacitidine). In certain embodiments, the cytidine analog is 5-aza-2′-deoxycytidine (decitabine). In certain embodiments, the cytidine analog is 5-azacytidine (5-azacitidine) or 5-aza-2′-deoxycytidine (decitabine). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-C R); N⁴-pentyloxy-carbonyl-5′-deoxy-5-fluorocytidine (Capecitabine); N⁴-octadecyl-cytarabine; or elaidic acid cytarabine. In certain embodiments, the cytidine analogs provided herein include any compound which is structurally related to cytidine or deoxycytidine and functionally mimics and/or antagonizes the action of cytidine or deoxycytidine.

Certain embodiments herein provide salts, cocrystals, solvates (e.g., hydrates), complexes, prodrugs, precursors, metabolites, and/or other derivatives of the cytidine analogs provided herein. For example, particular embodiments provide salts, cocrystals, solvates (e.g., hydrates), complexes, precursors, metabolites, and/or other derivatives of 5-azacytidine. Certain embodiments herein provide salts, cocrystals, and/or solvates (e.g., hydrates) of the cytidine analogs provided herein. Certain embodiments herein provide salts and/or solvates (e.g., hydrates) of the cytidine analogs provided herein. Certain embodiments provide cytidine analogs that are not salts, cocrystals, solvates (e.g., hydrates), or complexes of the cytidine analogs provided herein. For example, particular embodiments provide 5-azacytidine in a non-ionized, non-solvated (e.g., anhydrous), non-complexed form. Certain embodiments herein provide a mixture of two or more cytidine analogs provided herein.

In one embodiment, the compound used in the methods provided herein is a free base, or a pharmaceutically acceptable salt or solvate thereof. In one embodiment, the free base or the pharmaceutically acceptable salt or solvate is a solid. In another embodiment, the free base or the pharmaceutically acceptable salt or solvate is a solid in an amorphous form. In yet another embodiment, the free base or the pharmaceutically acceptable salt or solvate is a solid in a crystalline form. For example, particular embodiments provide 5-azacytidine in solid forms, which can be prepared, for example, according to the methods described in U.S. Pat. Nos. 6,943,249, 6,887,855 and 7,078,518, and U.S. Patent Application Publication Nos. 2005/027675 and 2006/247189, each of which is incorporated by reference herein in their entireties. In other embodiments, 5-azacytidine in solid forms can be prepared using other methods known in the art.

In one embodiment, the compound used in the methods provided herein is a pharmaceutically acceptable salt of the cytidine analog, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts.

Cytidine analogs provided herein may be prepared using synthetic methods and procedures referenced herein or otherwise available in the literature. For example, particular methods for synthesizing 5-azacytidine are disclosed, e.g., in U.S. Pat. No. 7,038,038 and references discussed therein, each of which is incorporated herein by reference. Other cytidine analogs provided herein may be prepared, e.g., using procedures known in the art, or may be purchased from a commercial source. In one embodiment, the cytidine analogs provided herein may be prepared in a particular solid form (e.g., amorphous or crystalline form). See, e.g., U.S. patent application Ser. No. 10/390,578, filed Mar. 17, 2003 and U.S. patent application Ser. No. 10/390,530, filed Mar. 17, 2003, both of which are incorporated herein by reference in their entireties. In other embodiments, methods of synthesis include methods as disclosed in U.S. Pat. No. 7,038,038; U.S. Pat. No. 6,887,855; U.S. Pat. No. 7,078,518; U.S. Pat. No. 6,943,249; and U.S. Ser. No. 10/823,394, all incorporated by reference herein in their entireties.

### III. Histone Deacetylase (HDAC) Inhibitors

Histone deacetylases (HDAC) are enzymes capable of removing the acetyl group bound to the lysine residues in the N-terminal portion of histones or in other proteins. HDACs can be subdivided into four classes, on the basis of structural homologies. Class I HDACs (HDAC 1, 2, 3 and 8) are similar to the RPD3 yeast protein and are located in the cell nucleus. Class II HDACs (HDAC 4, 5, 6, 7, 9 and 10) are similar to the HDA1 yeast protein and arc located both in the nucleus and in the cytoplasm. Class III HDACs are a structurally distinct form of NAD-dependent enzymes correlated with the SIR2 yeast protein. Class IV (HDAC 11) consists at the moment of a single enzyme having particular structural characteristics. The HDACs of classes I, II and IV are zinc enzymes and can be inhibited by various classes of molecule: hydroxamic acid derivatives, cyclic tetrapeptides, short-chain fatty acids, aminobenzamides, derivatives of electrophilic ketones, and the like. Class III HDACs are not inhibited by hydroxamic acids, and their inhibitors have structural characteristics different from those of the other classes.

The expression “histone deacetylase inhibitor” in relation to the present invention is to be understood as meaning any molecule of natural, recombinant or synthetic origin capable of inhibiting the activity of at least one of the enzymes classified as HDAC. In particular embodiments, an HDAC inhibitor inhibits enzymes of Class I and II.

Examples of HDAC inhibitors useful in the compositions and methods of the present invention include, but are not limited to, givinostat, entinostat, trichostatin A (TSA), Vorinostat (SAHA), Valproic Acid (VPA), romidepsin and MS-275. In a specific embodiment, the HDAC inhibitor is givinostat (ITF2357; diethyl-[6-(4-hydroxycarbamoyl-phenylcarbamoyloxymethyl)-naphthalen-2-yl methyl]-ammonium chloride). See, e.g., WO97/43251 (anhydrous form) and in WO2004/065355 (monohydrate crystal form).

HDAC inhibitors also include chidamide, panobinostat (Farydak, LBH589), belinosiat (PXD101), mocetinostat (MGCD0103), abexinostat (PCI-24781), SB939, resminostat (4SC-201),quisinostat (JNJ26481585), Kevetrin, CUDC-101 AR-42, CHR-2845, CHR-3996, 4SC-202, ACCT-1215. and ME-344.

Other examples of HDAC inhibitors include those described in the following patent applications: WO2004/092115, WO2005/019174, WO2003/076422, WO1997/043251, WO2006/010750, WO2006/003068, WO2002/030879, WO2002/022577, WO1993/007148, WO2008/033747, WO2004/069823, EP0847992 and WO2004/071400, the contents of which are incorporated herein by reference in their entirety.

### IV. Checkpoint Inhibitors

In another aspect, the present invention provides compositions and methods for treating solid tumors with a combination therapy including a checkpoint inhibitor.

In particular embodiments, the checkpoint inhibitor is a biologic therapeutic or a small molecule. In certain embodiments, the checkpoint inhibitor is a monoclonal antibody, a humanized antibody, a fully human antibody, a fusion protein or a combination thereof. In a particular aspect, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In certain embodiments, therapeutic agent is an immunostimulatory agent, a T cell growth factor, an interleukin, an antibody, a vaccine or a combination thereof. In a further aspect, the interleukin is IL-7 or IL-15. In a specific embodiment, the interleukin is glycosylated IL-7. In another embodiment, the vaccine is a dendritic cell vaccine.

In other embodiments, the checkpoint inhibitor is of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

Checkpoint inhibitors include any agent that blocks or inhibits the immune system or immune responses. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γΔ, and memory CD8⁺ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PDL1, PDL2, PD1, BTLA, HVEM, TIM3, GAL9, LAG3, VISTA, KIR, 2B4, CD160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Checkpoint protein ligands include, but are not limited to PD-L1, PD-L2, B7-H3, B7-H4, CD28, CD86 and TIM-3.

In one specific embodiment, the present invention covers the use of a specific class of checkpoint inhibitors that are drugs that block the interaction between immune checkpoint receptor programmed cell death protein 1 (PD-1) and its ligand PDL-1. See A. Mullard, “New checkpoint inhibitors ride the immunotherapy tsunami,” Nature Reviews: Drug Discovery (2013), 12:489-492. PD-1 is expressed on and regulates the activity of T-cells. Specifically, when PD-1 is unbound to PDL-1, the T-cells can engage and kill target cells. However, when PD-1 is bound to PDL-1 it causes the T-cells to cease engaging and killing target cells. Furthermore, unlike other checkpoints, PD-1 acts proximately. The PDLs are overexpressed directly on cancer cells which leads to increased binding to the PD-1 expressing T-cells.

As used herein, the term “PD-1 antibodies” refers to antibodies that antagonize the activity and/or proliferation of lymphocytes by agonizing PD-1. The term “antagonize the activity” relates to a decrease (or reduction) in lymphocyte proliferation or activity that is at least 5%, 10%, 20%, 30%, 40%, 50%, 60%, 70%, 80%, 90%, or more. The term “antagonize” may be used interchangeably with the terms “inhibitory” and “inhibit”. PD-1-mediated activity can be determined quantitatively using T cell proliferation assays as described herein. There are several PD-1 inhibitors currently being tested in clinical trials including CT-011, BMS 936558, BMS 936559, MK 3475, MPDL 3280A, AMP224, Medi 4736.

One aspect of the present disclosure provides checkpoint inhibitors which are antibodies that can act as agonists of PD-1, thereby modulating immune responses regulated by PD-1. In one embodiment, the anti-PD-1 antibodies can be antigen-binding fragments. Anti-PD-1 antibodies disclosed herein are able to bind to human PD-1 and agonize the activity of PD-1, thereby inhibiting the function of immune cells expressing PD-1.

In one specific embodiment, the present invention covers the use of a specific class of checkpoint inhibitor are drugs that inhibit CTLA-4. Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO2001/014424, the antibodies disclosed in PCT Publication No. WO2004/035607, the antibodies disclosed in U.S. Publication No. 2005/0201994, and the antibodies disclosed in granted European Patent No. EP1212422 B1. Additional CTLA-4 antibodies are described in U.S. Pat. Nos. 5,811,097, 5,855,887, 6,051,227, and 6,984,720; in PCT Publication Nos. WO01/14424 and WO00/37504; and in U.S. Publication Nos. 2002/0039581 and 2002/086014. Other anti-CTLA-4 antibodies that can be used in a method of the present invention include, for example, those disclosed in: WO98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

In one specific embodiment, the present invention covers the use of a specific class of checkpoint inhibitor are drugs that inhibit TIM-3. Blocking the activation of TIM-3 by a ligand, results in an increase in Thl cell activation. Furthermore, TIM-3 has been identified as an important inhibitory receptor expressed by exhausted CD8+ T cells. TIM-3 has also been reported as a key regulator of nucleic acid mediated antitumor immunity. In one example, TIM-3 has been shown to be upregulated on tumor-associated dendritic cells (TADCs).

### V. Pharmaceutical Compositions and Formulations

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the demethylating agent, HDAC inhibitor and/or checkpoint inhibitor are administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a demethylating agent and HDAC inhibitor and optionally a checkpoint inhibitor together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection.

Optimal precision in achieving concentrations of the therapeutic regimen (e.g., pharmaceutical compositions comprising a demethylating agent and an HDAC inhibitor in combination with another therapeutic agent such as a checkpoint inhibitor) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the EDso (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO00/67776, which is entirely expressly incorporated herein by reference.

### VI. Exemplary Methods of Use

In one embodiment, an effective amount of a demethylating agent, an HDAC inhibitor and/or a checkpoint inhibitor to be used is a therapeutically effective amount. In one embodiment, the amounts of the drugs to be used in the methods provided herein include an amount sufficient to cause improvement in at least a subset of patients with respect to symptoms, overall course of disease, or other parameters known in the art. Precise amounts for therapeutically effective amounts in the pharmaceutical compositions and methods will vary depending on the age, weight, disease, and condition of the patient, as well as the particular drug being administered.

In one embodiment, an HDAC inhibitor (e.g., givinostat, entinostat, romidepsin and the like) is administered intravenously. In one embodiment, the HDAC inhibitor is administered intravenously over a 1-6 hour period. In one embodiment, the HDAC inhibitor is administered intravenously over a 3-4 hour period. In one embodiment, the HDAC inhibitor is administered intravenously over a 5-6 hour period. In one embodiment, the HDAC inhibitor is administered intravenously over a 4 hour period.

In one embodiment, the HDAC inhibitor is administered in a dose ranging from 0.5 mg/m² to 28 mg/m². In one embodiment, the HDAC inhibitor is administered in a dose ranging from 0.5 mg/m² to 5 mg/m². In one embodiment, the HDAC inhibitor is administered in a dose ranging from 1 mg/m² to 25 mg/m². In one embodiment, the HDAC inhibitor is administered in a dose ranging from 1 mg/m² to 20 mg/m². In one embodiment, the HDAC inhibitor is administered in a dose ranging from 1 mg/m² to 15 mg/m². In one embodiment, the HDAC inhibitor is administered in a dose ranging from 2 mg/m² to 15 mg/m². In one embodiment, the HDAC inhibitor is administered in a dose ranging from 2 mg/m² to 12 mg/m². In one embodiment, the HDAC inhibitor is administered in a dose ranging from 4 mg/m² to 12 mg/m². In one embodiment, the HDAC inhibitor is administered in a dose ranging from 6 mg/m² to 12 mg/m². In one embodiment, the HDAC inhibitor is administered in a dose ranging from 8 mg/m² to 12 mg/m². In one embodiment, the HDAC inhibitor is administered in a dose ranging from 8 mg/m² to 10 mg/m². In one embodiment, the HDAC inhibitor is administered in a dose of about 8 mg/m². In one embodiment, the HDAC inhibitor is administered in a dose of about 9 mg/m². In one embodiment, the HDAC inhibitor is administered in a dose of about 10 mg/m². In one embodiment, the HDAC inhibitor is administered in a dose of about 11 mg/m². In one embodiment, the HDAC inhibitor is administered in a dose of about 12 mg/m². In one embodiment, the HDAC inhibitor is administered in a dose of about 13 mg/m². In one embodiment, the HDAC inhibitor is administered in a dose of about 14 mg/m². In one embodiment, the HDAC inhibitor is administered in a dose of about 15 mg/m².

In one embodiment, the HDAC inhibitor is administered in a dose of 14 mg/m² over a 4 hour iv infusion on days 1, 8 and 15 of the 28 day cycle. In one embodiment, the cycle is repeated every 28 days.

In one embodiment, increasing doses of the HDAC inhibitor are administered over the course of a cycle. In one embodiment, the dose of about 8 mg/m² followed by a dose of about 10 mg/m², followed by a dose of about 12 mg/m² is administered over a cycle.

In one embodiment, the HDAC inhibitor is administered orally. In one embodiment, the HDAC inhibitor is administered in a dose ranging from 10 mg/m² to 300 mg/m². In one embodiment, the HDAC inhibitor is administered in a dose ranging from 15 mg/m² to 250 mg/m². In one embodiment, the HDAC inhibitor is administered in a dose ranging from 20 mg/m² to 200 mg/m². In one embodiment, the HDAC inhibitor is administered in a dose ranging from 25 mg/m² to 150 mg/m². In one embodiment, the HDAC inhibitor is administered in a dose ranging from 25 mg/m² to 100 mg/m². In one embodiment, the HDAC inhibitor is administered in a dose ranging from 25 mg/m² to 75 mg/m².

In one embodiment, the HDAC inhibitor is administered orally on a daily basis. In one embodiment, the HDAC inhibitor is administered orally every other day. In one embodiment, the HDAC inhibitor is administered orally every third, fourth, fifth, or sixth day. In one embodiment, the HDAC inhibitor is administered orally every week. In one embodiment, the HDAC inhibitor is administered orally every other week. Merck's ZOLINZA® (vorinostat) is administered 400 mg orally once daily with food.

In particular embodiments, the demethylating agent is administered by, e.g., intravenous (IV), subcutaneous (SC) or oral routes. Certain embodiments herein provide co-administration of the demethylating agent with one or more additional active agents to provide a synergistic therapeutic effect in subjects in need thereof. The co-administered agent(s) may be a cancer therapeutic agent, as described herein. In certain embodiments, the co-administered agent(s) may be dosed, e.g., orally or by injection (e.g., IV or SC).

Certain embodiments herein provide methods for treating solid tumors comprising administering the demethylating agent using, e.g., IV, SC and/or oral administration methods. In certain embodiments, treatment cycles comprise multiple doses administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. For example, in certain embodiments, the amount of the demethylating agent administered in the methods provided herein may range, e.g., between about 30 mg/m²/day and about 2,000 mg/m²/day, between about 100 mg/m²/day and about 1,000 mg/m²/day, between about 100 mg/m²/day and about 500 mg/m²/day, between about 30 mg/m²/day and about 500 mg/m²/day, between about 30 mg/m²/day and about 200 mg/m²/day, between about 30 mg/m²/day and about 100 mg/m²/day, between about 30 mg/m²/day and about 75 mg/m²/day, or between about 120 mg/m²/day and about 250 mg/m²/day. In certain embodiments, particular dosages are, e.g., about 30 mg/m²/day, about 40 mg/m²/day, about 50 mg/m²/day, about 60 mg/m²/day, about 75 mg/m²/day, about 80 mg/m²/day, about 100 mg/m²/day, about 120 mg/m²/day, about 140 mg/m²/day, about 150 mg/m²/day, about 180 mg/m²/day, about 200 mg/m²/day, about 220 mg/m²/day, about 240 mg/m²/day, about 250 mg/m²/day, about 260 mg/m²/day, about 280 mg/m²/day, about 300 mg/m²/day, about 320 mg/m²/day, about 350 mg/m²/day, about 380 mg/m²/day, about 400 mg/m²/day, about 450 mg/m²/day, or about 500 mg/m²/day. In certain embodiments, particular dosages are, e.g., up to about 30 mg/m²/day, up to about 40 mg/m²/day, up to about 50 mg/m²/day, up to about 60 mg/m²/day, up to about 70 mg/m²/day, up to about 80 mg/m²/day, up to about 90 mg/m²/day, up to about 100 mg/m²/day, up to about 120 mg/m²/day, up to about 140 mg/m²/day, up to about 150 mg/m²/day, up to about 180 mg/m²/day, up to about 200 mg/m²/day, up to about 220 mg/m²/day, up to about 240 mg/m²/day, up to about 250 mg/m²/day, up to about 260 mg/m²/day, up to about 280 mg/m²/day, up to about 300 mg/m²/day, up to about 320 mg/m²/day, up to about 350 mg/m²/day, up to about 380 mg/m²/day, up to about 400 mg/m²/day, up to about 450 mg/m²/day, up to about 500 mg/m²/day, up to about 750 mg/m²/day, or up to about 1000 mg/m²/day. In a specific non-limiting embodiment, the dose of the demethylating agent is about 40 mg/m².

In one embodiment, the amount of the demethylating agent administered in the methods provided herein may range, e.g., between about 5 mg/day and about 2,000 mg/day, between about 10 mg/day and about 2,000 mg/day, between about 20 mg/day and about 2,000 mg/day, between about 50 mg/day and about 1,000 mg/day, between about 100 mg/day and about 1,000 mg/day, between about 100 mg/day and about 500 mg/day, between about 150 mg/day and about 500 mg/day, or between about 150 mg/day and about 250 mg/day. In certain embodiments, particular dosages are, e.g., about 10 mg/day, about 20 mg/day, about 50 mg/day, about 75 mg/day, about 100 mg/day, about 120 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1,000 mg/day, about 1,200 mg/day, or about 1,500 mg/day. In certain embodiments, particular dosages are, e.g., up to about 10 mg/day, up to about 20 mg/day, up to about 50 mg/day, up to about 75 mg/day, up to about 100 mg/day, up to about 120 mg/day, up to about 150 mg/day, up to about 200 mg/day, up to about 250 mg/day, up to about 300 mg/day, up to about 350 mg/day, up to about 400 mg/day, up to about 450 mg/day, up to about 500 mg/day, up to about 600 mg/day, up to about 700 mg/day, up to about 800 mg/day, up to about 900 mg/day, up to about 1,000 mg/day, up to about 1,200 mg/day, or up to about 1,500 mg/day.

In one embodiment, the amount of the demethylating agent in the pharmaceutical composition or dosage form provided herein may range, e.g., between about 5 mg and about 2,000 mg, between about 10 mg and about 2,000 mg, between about 20 mg and about 2,000 mg, between about 30 mg and about 1,000 mg, between about 30 mg and about 500 mg, between about 30 mg and about 250 mg, between about 100 mg and about 500 mg, between about 150 mg and about 500 mg, or between about 150 mg and about 250 mg. In certain embodiments, particular amounts are, e.g., about 10 mg, about 20 mg, about 30 mg, about 40 mg, about 50 mg, about 75 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 1,200 mg, or about 1,500 mg. In certain embodiments, particular amounts are, e.g., up to about 10 mg, up to about 20 mg, up to about 30 mg, up to about 40 mg, up to about 50 mg, up to about 75 mg, up to about 100 mg, up to about 120 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1,000 mg, up to about 1,200 mg, or up to about 1,500 mg.

In one embodiment, depending on the disease to be treated and the subject's condition, the demethylating agent may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The demethylating agent may be formulated, alone or together with one or more active agent(s), in suitable dosage unit with pharmaceutically acceptable excipients, carriers, adjuvants and vehicles, appropriate for each route of administration. In one embodiment, the demethylating agent is administered orally. In another embodiment, the demethylating agent is administered parenterally. In yet another embodiment, the demethylating agent is administered intravenously.

In one embodiment, the demethylating agent can be delivered as a single dose such as, e.g., a single bolus injection, or oral tablets or pills; or over time such as, e.g., continuous infusion over time or divided bolus doses over time. In one embodiment, the demethylating agent can be administered repetitively if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. See, e.g., Response Evaluation Criteria in Solid Tumors (RECIST) Guidelines, Journal of the National Cancer Institute 92(3): 205-216 (2000). Stable disease or lack thereof is determined by methods known in the art such as evaluation of patient's symptoms, physical examination, visualization of the tumor that has been imaged using X-ray, CAT, PET, or MRI scan and other commonly accepted evaluation modalities.

In one embodiment, the demethylating agent can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, the demethylating agent is administered daily, for example, once or more than once each day for a period of time. In one embodiment, the demethylating agent is administered daily for an uninterrupted period of at least 7 days, in some embodiments, up to 52 weeks. In one embodiment, the demethylating agent is administered intermittently, i.e., stopping and starting at either regular or irregular intervals.

In one embodiment, the demethylating agent is administered for one to six days per week. In one embodiment, the demethylating agent is administered in cycles (e.g., daily administration for two to eight consecutive weeks, then a rest period with no administration for up to one week; or e.g., daily administration for one week, then a rest period with no administration for up to three weeks). In one embodiment, the demethylating agent is administered on alternate days. In one embodiment, the demethylating agent is administered in cycles (e.g., administered daily or continuously for a certain period interrupted with a rest period).

In one embodiment, the frequency of administration ranges from about daily to about monthly. In certain embodiments, the demethylating agent is administered once a day, twice a day, three times a day, four times a day, once every other day, twice a week, once every week, once every two weeks, once every three weeks, or once every four weeks. In one embodiment, the demethylating agent is administered once a day. In another embodiment, the demethylating agent is administered twice a day. In yet another embodiment, the demethylating agent is administered three times a day. In still another embodiment, the demethylating agent is administered four times a day.

In one embodiment, the demethylating agent is administered once per day from one day to six months, from one week to three months, from one week to four weeks, from one week to three weeks, or from one week to two weeks. In certain embodiments, the demethylating agent is administered once per day for one week, two weeks, three weeks, or four weeks. In one embodiment, the demethylating agent is administered once per day for one week. In another embodiment, the demethylating agent is administered once per day for two weeks. In yet another embodiment, the demethylating agent is administered once per day for three weeks. In still another embodiment, the demethylating agent is administered once per day for four weeks.

In one embodiment, the demethylating agent is administered once per day for about 1 week, about 2 weeks, about 3 weeks, about 4 weeks, about 6 weeks, about 9 weeks, about 12 weeks, about 15 weeks, about 18 weeks, about 21 weeks, or about 26 weeks. In certain embodiments, the demethylating agent is administered intermittently. In certain embodiments, the demethylating agent is administered intermittently in the amount of between about 30 mg/m²/day and about 2,000 mg/m²/day. In certain embodiments, the demethylating agent is administered continuously. In certain embodiments, the demethylating agent is administered continuously in the amount of between about 30 mg/m²/day and about 1,000 mg/m²/day.

In certain embodiments, the demethylating agent is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, the demethylating agent is administered to a patient in cycles. In one embodiment, a method provided herein comprises administering the demethylating agent in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or greater than about 30 cycles.

In one embodiment, the demethylating agent is administered to a patient at a dose provided herein over a cycle of 28 days which consists of a 7-day treatment period and a 21-day resting period. In one embodiment, the demethylating agent is administered to a patient at a dose provided herein each day from day 1 to day 7, followed with a resting period from day 8 to day 28 with no administration of the demethylating agent. In one embodiment, the demethylating agent is administered to a patient in cycles, each cycle consisting of a 7-day treatment period followed with a 21-day resting period. In particular embodiments, the demethylating agent is administered to a patient at a dose of about 50, about 60, about 70, about 75, about 80, about 90, or about 100 mg/m²/d, for 7 days, followed with a resting period of 21 days. In one embodiment, the demethylating agent is administered intravenously. In one embodiment, the demethylating agent is administered subcutaneously.

In other embodiments, the demethylating agent is administered orally in cycles.

Accordingly, in one embodiment, the demethylating agent is administered daily in single or divided doses for about one week, about two weeks, about three weeks, about four weeks, about five weeks, about six weeks, about eight weeks, about ten weeks, about fifteen weeks, or about twenty weeks, followed by a rest period of about 1 day to about ten weeks. In one embodiment, the methods provided herein contemplate cycling treatments of about one week, about two weeks, about three weeks, about four weeks, about five weeks, about six weeks, about eight weeks, about ten weeks, about fifteen weeks, or about twenty weeks. In some embodiments, the demethylating agent is administered daily in single or divided doses for about one week, about two weeks, about three weeks, about four weeks, about five weeks, or about six weeks with a rest period of about 1, 3, 5, 7, 9, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, the methods provided herein comprise: i) administering to the subject a first daily dose of the demethylating agent; ii) optionally resting for a period of at least one day where the demethylating agent is not administered to the subject; iii) administering a second dose of the demethylating agent to the subject; and iv) repeating steps ii) to iii) a plurality of times. In certain embodiments, the first daily dose is between about 30 mg/m²/day and about 2,000 mg/m²/day. In certain embodiments, the second daily dose is between about 30 mg/m²/day and about 2,000 mg/m²/day. In certain embodiments, the first daily dose is higher than the second daily dose. In certain embodiments, the second daily dose is higher than the first daily dose. In one embodiment, the rest period is 2 days, 3 days, 5 days, 7 days, 10 days, 12 days, 13 days, 14 days, 15 days, 17 days, 21 days, or 28 days. In one embodiment, the rest period is at least 2 days and steps ii) through iii) are repeated at least three times. In one embodiment, the rest period is at least 2 days and steps ii) through iii) are repeated at least five times. In one embodiment, the rest period is at least 3 days and steps ii) through iii) are repeated at least three times. In one embodiment, the rest period is at least 3 days and steps ii) through iii) are repeated at least five times. In one embodiment, the rest period is at least 7 days and steps ii) through iii) are repeated at least three times. In one embodiment, the rest period is at least 7 days and steps ii) through iii) are repeated at least five times. In one embodiment, the rest period is at least 14 days and steps ii) through iii) are repeated at least three times. In one embodiment, the rest period is at least 14 days and steps ii) through iii) are repeated at least five times. In one embodiment, the rest period is at least 21 days and steps ii) through iii) are repeated at least three times. In one embodiment, the rest period is at least 21 days and steps ii) through iii) are repeated at least five times. In one embodiment, the rest period is at least 28 days and steps ii) through iii) are repeated at least three times. In one embodiment, the rest period is at least 28 days and steps ii) through iii) are repeated at least five times. In one embodiment, the methods provided herein comprise: i) administering to the subject a first daily dose of the demethylating agent for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; iii) administering to the subject a second daily dose of the demethylating agent for 1, 2, 3, 4, 5, 6, 7, 8, 9, 10, 11, 12, 13, or 14 days; and iv) repeating steps ii) to iii) a plurality of times. In one embodiment, the methods provided herein comprise: i) administering to the subject a daily dose of the demethylating agent for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; and iii) repeating steps i) to ii) a plurality of times. In one embodiment, the methods provided herein comprise: i) administering to the subject a daily dose of the demethylating agent for 7 days; ii) resting for a period of 21 days; and iii) repeating steps i) to ii) a plurality of times. In one embodiment, the daily dose is between about 30 mg/m²/day and about 2,000 mg/m²/day. In one embodiment, the daily dose is between about 30 mg/m²/day and about 1,000 mg/m²/day. In one embodiment, the daily dose is between about 30 mg/m²/day and about 500 mg/m²/day. In one embodiment, the daily dose is between about 30 mg/m²/day and about 200 mg/m²/day. In one embodiment, the daily dose is between about 30 mg/m²/day and about 100 mg/m²/day.

In certain embodiments, the demethylating agent is administered continuously for between about 1 and about 52 weeks. In certain embodiments, the demethylating agent is administered continuously for about 1, 2, 3, 4, 5, 6, 7, 8, 9, 10, 11, or 12 months. In certain embodiments, the demethylating agent is administered continuously for about 14, about 28, about 42, about 84, or about 112 days. It is understood that the duration of the treatment may vary with the age, weight, and condition of the subject being treated, and may be determined empirically using known testing protocols or according to the professional judgment of the person providing or supervising the treatment. The skilled clinician will be able to readily determine, without undue experimentation, an effective drug dose and treatment duration, for treating an individual subject having a particular type of cancer.

In one embodiment, pharmaceutical compositions may contain sufficient quantities of the demethylating agent to provide a daily dosage of about 10 to 150 mg/m² (based on patient body surface area) or about 0.1 to 4 mg/kg (based on patient body weight) as single or divided (2-3) daily doses. In one embodiment, dosage is provided via a seven-day administration of 75 mg/m² subcutaneously, once every twenty-eight days, for as long as clinically necessary. In one embodiment, dosage is provided via a seven-day administration of 100 mg/m² subcutaneously, once every twenty-eight days, for as long as clinically necessary. In one embodiment, up to 4, up to 5, up to 6, up to 7, up to 8, up to 9 or more 28-day cycles are administered. Other methods for providing an effective amount of the demethylating agent are disclosed in, for example, “Colon-Targeted Oral Formulations of Cytidine Analogs”, U.S. Ser. No. 11/849,958, and “Oral Formulations of Cytidine Analogs and Methods of Use Thereof”, U.S. Ser. No. 12/466,213, both of which are incorporated by reference herein in their entireties.

In particular embodiments, the number of cycles administered is, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, at least 30, at least 32, at least 34, at least 36, at least 38, at least 40, at least 42, at least 44, at least 46, at least 48, or at least 50 cycles of the demethylating agent treatment. In particular embodiments, the treatment is administered, e.g., 1, 2, 3, 4, 5, 6, 7, 8, 9, 10, 11, 12, 13, or 14 days out of a 28-day period. In particular embodiments, the demethylating agent dose is, e.g., at least 10 mg/day, at least 20 mg/day, at least 30 mg/day, at least 40 mg/day, at least 50 mg/day, at least 55 mg/day, at least 60 mg/day, at least 65 mg/day, at least 70 mg/day, at least 75 mg/day, at least 80 mg/day, at least 85 mg/day, at least 90 mg/day, at least 95 mg/day, or at least 100 mg/day.

In particular embodiments, the dosing is performed, e.g., subcutaneously or intravenously. In particular embodiments, the contemplated specific the demethylating agent dose is, e.g., at least 30 mg/m²/day, at least 40 mg/m²/day, at least 50 mg/m²/day, at least 60 mg/m²/day, at least 70 mg/m²/day, at least 75 mg/m²/day, at least 80 mg/m2/day, at least 90 mg/m²/day, or at least 100 mg/m²/day. One particular embodiment herein provides administering the treatment for 7 days out of each 28-day period. One particular embodiment herein provides a dosing regimen of 75 mg/m² subcutaneously or intravenously, daily for 7 days. One particular embodiment herein provides a dosing regimen of 100 mg/m² subcutaneously or intravenously, daily for 7 days.

In one embodiment, the HDAC inhibitor and the demethylating agent (and optionally the checkpoint inhibitor) are administered intravenously. In one embodiment, the combination is administered intravenously over a 1-6 hour period. In one embodiment, the combination is administered intravenously over a 3-4 hour period. In one embodiment, the combination is administered intravenously over a 5-6 hour period. In one embodiment, the combination is administered intravenously over a 4 hour period.

In one embodiment, a combination with increasing doses of the HDAC inhibitor is administered over the course of a cycle. In one embodiment, the dose of about 8 mg/m² followed by a dose of about 10 mg/m², followed by a dose of about 12 mg/m² of the HDAC inhibitor is administered over a cycle.

In one embodiment, the HDAC inhibitor is administered intravenously and the demethylating agent (and optionally the checkpoint inhibitor) is administered subcutaneously. In one embodiment, the HDAC inhibitor is administered intravenously and the demethylating agent (and optionally the checkpoint inhibitor) is administered orally. In one embodiment, the HDAC inhibitor and the demethylating agent (and optionally the checkpoint inhibitor) are administered orally.

In one embodiment, the demethylating agent is administered daily based on 7 to 14 days administration every 28-day cycle in a single or divided doses in a four to forty week period with a rest period of about a week or two weeks.

In one embodiment, the demethylating agent is administered daily and continuously for four to forty weeks at a dose of from about 10 to about 150 mg/m² followed by a break of one or two weeks. In a particular embodiment, the demethylating agent is administered in an amount of from about 0.1 to about 4.0 mg/day for four to forty weeks, with one week or two weeks of rest in a four or six week cycle.

In one embodiment, the demethylating agent is administered intravenously to patients with solid tumors in an amount of from about 0.1 to about 4.0 mg per day for about 7 to about 14 days followed by about 14 to about 21 days of rest in a 28 day cycle combined with the HDAC inhibitor administered intravenously in a dose of about 0.5 mg/m² to about 28 mg/m² administered on days 1, 8 and 15 of the 28 day cycle.

In one embodiment, the demethylating agent is administered intravenously to patients with solid tumors in an amount of from about 0.10 to about 4.0 mg per day for about 7 to about 14 days followed by about 14 to about 21 day of rest in a 28 day cycle combined with the HDAC inhibitor administered orally in a dose of about 10 mg/m² to about 300 mg/m² administered on days 1, 8 and 15 of the 28 day cycle.

In one embodiment, the demethylating agent is administered subcutaneously to patients with solid tumors in an amount of from about 0.10 to about 4.0 mg per day for about 7 to about 14 days followed by about 14 to about 21 day of rest in a 28 day cycle combined with the HDAC inhibitor administered intravenously in a dose of about 10 mg/m² to about 300 mg/m² administered on days 1, 8 and 15 of the 28 day cycle.

In one embodiment, the demethylating agent is administered subcutaneously to patients with solid tumors in an amount of from about 0.10 to about 4.0 mg per day for about 7 to about 14 days followed by about 14 to about 21 day of rest in a 28 day cycle combined with the HDAC inhibitor administered orally in a dose of about 10 mg/m² to about 300 mg/m² administered on days 1, 8 and 15 of the 28 day cycle.

In one embodiment, the demethylating agent is administered orally to patients with solid tumors in an amount of from about 0.10 to about 4.0 mg per day for about 7 to about 14 days followed by about 14 to about 21 day of rest in a 28 day cycle combined with the HDAC inhibitor administered orally in a dose of about 10 mg/m² to about 300 mg/m² administered on days 1, 8 and 15 of the 28 day cycle.

In one embodiment, the demethylating agent and the HDAC inhibitor (and optionally the checkpoint inhibitor) are administered intravenously, with administration of the HDAC inhibitor occurring 30 to 60 minutes prior to the demethylating agent during a cycle of four to forty weeks. In another embodiment, the demethylating agent is administered subcutaneously and the HDAC inhibitor (and optionally the checkpoint inhibitor) is administered by intravenous infusion. In another embodiment, the demethylating agent is administered subcutaneously and the HDAC inhibitor (and optionally the checkpoint inhibitor) is administered orally. In yet another embodiment, the demethylating agent and the HDAC inhibitor (and optionally the checkpoint inhibitor) are administered orally.

In one embodiment, the demethylating agent and the HDAC inhibitor (and optionally the checkpoint inhibitor) are administered intravenously, with administration of the demethylating agent occurring 30 to 60 minutes prior to the HDAC inhibitor, during a cycle of four to forty weeks. In another embodiment, the demethylating agent is administered subcutaneously and the HDAC inhibitor (and optionally the checkpoint inhibitor) is administered by intravenous infusion. In another embodiment, the demethylating agent is administered subcutaneously and the HDAC inhibitor (and optionally the checkpoint inhibitor) is administered orally. In yet another embodiment, the demethylating agent and the HDAC inhibitor (and optionally the checkpoint inhibitor) are administered orally.

In one embodiment, the demethylating agent and the HDAC inhibitor (and optionally the checkpoint inhibitor) are administered intravenously, simultaneously, during a cycle of four to forty weeks. In another embodiment, the demethylating agent is administered subcutaneously and the HDAC inhibitor (and optionally the checkpoint inhibitor) is administered by intravenous infusion. In another embodiment, the demethylating agent is administered subcutaneously and the HDAC inhibitor (and optionally the checkpoint inhibitor) is administered orally. In yet another embodiment, the demethylating agent and the HDAC inhibitor (and optionally the checkpoint inhibitor) are administered orally.

Any suitable daily dose of a checkpoint inhibitor is contemplated for use with the compositions, dosage forms, and methods disclosed herein. Daily dose of the checkpoint inhibitor depends on multiple factors, the determination of which is within the skills of one of skill in the art. For example, the daily dose of the checkpoint inhibitor depends on the strength of the checkpoint inhibitor. Weak immune checkpoint inhibitors will require higher daily doses than moderate immune checkpoint inhibitors, and moderate immune checkpoint inhibitors will require higher daily doses than strong immune checkpoint inhibitors. For example, Merck's pembrolizumab (Keytruda) is approved for 2 mg/kg iv over 30 minutes every three weeks (50 mg lyophilized power). Nivolumab (OPDVO) is administered 3 mg/kg iv over 60 minutes every 2 weeks (injection dosage form: 40 mg/4 ml and 100 mg/10/ml in single use vial). Ipilimumab (YERVOY) is administered 3 mg/kg iv over 90 minutes every 3 weeks for a total of 4 doses (dosage form: 50 mg/10 ml, 200 mg/40 ml).

### VII. Kits

In other embodiments, kits are provided. Kits according to the invention include package(s) comprising compounds or compositions of the invention. In some embodiments, kits comprise a demethylating agent, a HDAC inhibitor, and/or a checkpoint inhibitor. A kit can comprise a demethylating agent and a HDAC inhibitor. In other embodiments, the kit comprises a demethylating agent, a HDAC inhibitor, and a checkpoint inhibitor. The kit can also comprise a dose form for a demethylating agent that can be used to prime the patient. In certain embodiments, the kit further comprises a demethylating agent and HDAC inhibitor composition.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions of the invention to a patient. Kits also can comprise instructions for approved uses of compounds herein by regulatory agencies, such as the United States Food and Drug Administration. Kits can also contain labeling or product inserts for the compounds. The package(s) and/or any product insert(s) may themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

1. A method for treating a solid tumor in a patient having cancer comprising the step of administering to the patient a demethylating agent, a histone deacetylase (HDAC) inhibitor and a checkpoint inhibitor.

2. The method of claim 1, wherein the demethylating agent is 5-azacytidine (AZA) or 5-azadeoxycytidine (DAC).

3. The method of claim 1, wherein the HDAC inhibitor is givinostat or entinostat.

4. The method of claim 1, wherein the HDAC inhibitor inhibits class I and class II histone deacetylases.

5. The method of claim 1, wherein the checkpoint inhibitor is an anti-PD1 antibody, an anti-PDL-1 antibody or an anti-CTLA4 antibody.

6. The method of claim 5, wherein the anti-PD1 antibody is nivolumab or pembrolizumab.

7. The method of claim 5, wherein the anti-PDL-1 antibody is Medi4736 or MPDL3280A.

8. The method of claim 5, wherein the anti-CTLA4 antibody is tremelimumab.

9. The method of claim 1, wherein the patient is administered a demethylating agent prior to the step of administering a demethylating agent, HDAC inhibitor and a checkpoint inhibitor.

10. A method for treating a solid tumor in a patient having cancer comprising the step of administering to the patient a demethylating agent and an HDAC inhibitor.

11. The method of claim 10, further comprising administering a checkpoint inhibitor.

12. The method of claim 10, further comprising the step of administering a demethylating agent prior to the step of administering a demethylating agent and an HDAC inhibitor.

13. A method for treating a solid tumor in a patient comprising the step of administering:
(a) AZA or DAC;
(b) givinostat or entinostat; and
(c) nivolumab, pembrolizumab, Medi4736, MPDL3280A or tremelimumab.

14. A pharmaceutical composition comprising a demethylating agent and an HDAC inhibitor.

15. The pharmaceutical composition of claim 14, further comprising a checkpoint inhibitor.

16. The pharmaceutical composition of claim 14, wherein the demethylating agent is AZA or DAC.

17. The pharmaceutical composition of claim 14, wherein the HDAC inhibitor is givinostat or entinostat.

18. The pharmaceutical composition of claim 14, wherein the demethylating agent is AZA or DAC and the HDAC inhibitor is givinostat or entinostat.

19. The pharmaceutical composition of claim 14, wherein the demethylating agent is AZA and the HDAC inhibitor is givinostat.

20. A pharmaceutical composition comprising a demethylating agent, an HDAC inhibitor and a checkpoint inhibitor.

21. A composition comprising a demethylating agent, an HDAC inhibitor and a checkpoint inhibitor for use in treating solid tumors.

22. A kit comprising a demethylating agent, an HDAC inhibitor, a checkpoint inhibitor and instructions for administration to treat solid tumors.
